(19) United States
(12) Patent Application Publication
(10) Pub. No.: US 2009/0202633 A1
(43) Pub. Date: Aug. 13, 2009

(54) Extended release formulations of guaifenesin

(76) Inventors: Siva Ramakrishna Velaga, Hyderabad (IN); Saravanan Kannusamy, Hyderabad (IN); Madhu Kandukuri, Hyderabad (IN); Venkata Ram Mohan Rao Visinigiri, Hyderabad (IN); Nagaprasad Vishnubhotta, Hyderabad (IN); Sivakumaran Meenakshisunderam, Hyderabad (IN)

Correspondence Address: Jay R. Akhave, 2058 N Mills Ave # 612, Claremont, CA, 91711, US

(21) Appl. No.: 12/319,189

(22) Filed: Jan. 2, 2009

(30) Foreign Application Priority Data
Jan. 3, 2008 (IN) 27/CHE/2008

(51) Int. Cl. A61K 31/485; A61K 31/075; A61K 31/4402; A61K 31/137; A61K 31/439; A61P 11/02; A61K 9/22

(52) U.S. Cl. 424/468; 514/718; 514/282; 514/357; 514/646; 514/289

(57) ABSTRACT

The present invention relates to extended release formulations comprising expectorant. More particularly, the present invention relates to extended release formulations comprising guaifenesin. The present invention also relates to a process for the preparation of extended release formulations comprising guaifenesin.

### FIELD OF THE INVENTION

The present invention relates to extended release formulations comprising expectorant. More particularly, the present invention relates to extended release formulations comprising guaifenesin.

The present invention also relates to a process for the preparation of extended release formulations comprising guaifenesin.

### BACKGROUND OF THE INVENTION

Guaifenesin is a drug derived from the Guaiacum officinale plant, which has yielded medicinal compounds of some form since the 1500s. Guaifenesin is chemically known as 3-(2-methoxyphenoxy)-1,2-propanediol. Guaifenesin is soluble in water and has bitter taste. It is an expectorant usually taken orally in acute respiratory tract infections. It is available as over-the-counter medicine for cough and cold in the form of tablets, solution and extended release tablet and is also available in combination with pseudoephedrine, dextromethorphan, acetaminophen and phenylephrine as tablets, capsules, extended release tablet, delayed release capsule, oral solution.

Guaifenesin is readily absorbed from the intestinal tract and is rapidly metabolized and excreted in urine. It has a typical plasma half-life of approximately one hour. Because of the rapid metabolism and excretion, typical immediate release dosage forms of guaifenesin are generally administered three times within 12 hours to maintain adequate bioavailability and to achieve therapeutic effect, which ultimately reduce the patient compliance.

To overcome the above disadvantages, extended release formulations were developed which can permit once in 12 hour dosing by maintaining a stable drug plasma concentration for an extended period of time. The extended release formulation reduces the side effects associated with the frequent administrations, which ultimately enhance the patient compliance.

The extended release tablets of guaifenesin and its combination with other drugs are well known and are commercially available under various brand names in the US for past few years. In 2002, FDA determined this DESI (Drug Efficacy Study Implementation) product to be a safety hazard to children. The manufacturers were told to stop manufacturing by November 2003 unless they received FDA approval. In July 2002 FDA approved NDA filed by Adams Laboratories for Guaifenesin extended release tablets under the brand name Mucinex®. FDA has also approved the combination products of Gauifenesin with dextromethorphan hydrobromide and pseudoephedrine under the names Mucinex DM® and Mucinex D® in 2004.

The FDA approved extended release tablets of Mucinex, Mucinex D® and Mucinex DM® are available as bilayered tablets with both immediate and modified release layers and contain carbomer, hydroxypropylmethylcellulose, magnesium stearate, microcrystalline cellulose, sodium starch glycolate and dyes as inactive ingredients.

Mucinex® extended release tablets contain 600 and 1200 mg of guaifenesin in both immediate and modified release layers.

Mucinex D® extended release tablets contains 600/60 & 1200/120 mg of guaifenesin & pseudoephedrine as active ingredients with guaifenesin in immediate release layer and both guaifenesin and pseudoephedrine in modified release layer.

Mucinex DM® extended release tablets contains 600/30 & 1200/60 mg of guaifenesin & dextromethorphan, with both immediate and modified release layers, each containing the two drugs.

The technology and composition of commercially available Mucinex® extended release tablets is disclosed in U.S. Pat. Nos. 6,372,252, 6,955,821. These patents disclose modified release tablets having two portions, wherein a first portion comprises a first quantity of guaifenesin in an immediate release form and a second portion comprises a second quantity of guaifenesin and a release-delaying matrix comprising a hydrophilic polymer and a water-insoluble polymer wherein the weight ratio of said hydrophilic polymer to said water-insoluble polymer is in the range of from about 1:1 to about 9:1.

Apart from the above patents, U.S. Pat. No. 4,798,725, U.S. Pat. No. 4,935,242, U.S. Pat. No. 5,133,974, U.S. Pat. No. 5,445,829, U.S. Pat. No. 6,416,786, U.S. Pat. No. 6,699,502, describe compositions that are suitable for extended release formulations of expectorant and its combination with decongestants.

Further, the following patents/patent publications disclose the guaifenesin formulations.

US 2003/0012820 discloses guaifenesin composition, comprising guaifenesin, binder, solubilizer and/or a disintegrant, glidant, and a lubricant and being in the form of particles.

US 2005/0266032 discloses a multi-layered tablet which comprises at least a first layer and a second layer, wherein the first layer comprises a first drug which is selected from decongestants, antitussives, expectorants, analgesics and antihistamines and the second layer comprises a second drug which is selected from decongestants, antitussives, expectorants, analgesics and antihistamines and is different from the first drug.

US 2005/0095288 discloses an oral pharmaceutical tablet formulation comprising a compressed matrix core comprising an expectorant, a decongestant and a controlled release coating on the core and finally atleast one coating applied to the controlled release coating to allow for the immediate release of the expectorants or decongestants.

US 2006/0257473 discloses an extended release tablet formulated as a single layered tablet or portion that provides a combination of immediate and extended release of active drugs comprising a granulation of guaifenesin, hydrophilic polymer and a water-insoluble, non-swellable particulate channeling agent such as colloidal silicon dioxide.

The above prior art references disclose various extended release formulations of guaifenesin such as multiple layer or single layer or coated technology. However, still there is a need to develop extended release formulations of guaifenesin avoiding the disadvantages associated with multilayered dosage forms like layer separation, insufficient hardness, inaccurate individual layer weight control and requirement of advanced technique etc.

With the aim of developing a cost-effective and bioequivalent extended release formulations of guaifenesin, the inventors of the present invention developed single layered matrix tablets comprising at least one hydrophilic polymer in intra and extra granular portion, which is capable of sustaining the therapeutic effects of guaifenesin for at least 12 hours.

### OBJECTIVE OF THE PRESENT INVENTION

Accordingly, the main objective of the present invention is to provide extended release formulation comprising guaifenesin.

Yet another objective of the present invention is to provide extended release formulation comprising guaifenesin in such a way that it will comply with the reference product in terms of in vivo parameters like Cₘₐₓ, tₘₐₓ, and AUC and in vitro parameters like dissolution etc.

Yet another objective of the present invention is to provide process for the preparation of extended release formulation comprising guaifenesin.

### SUMMARY OF THE INVENTION

Accordingly, the main embodiment of the present invention is to provide single layer extended release matrix tablet formulation comprising

i) intragranular portion comprising about 60% w/w of guaifenesin, at least one hydrophilic polymer, optionally a water insoluble polymer and
ii) extragranular portion comprising hydrophilic polymer and optionally guaifenesin,
wherein the ratio of hydrophilic polymer in the intra and extra granular portions is about 1:1 to about 1:10.

### DETAILED DESCRIPTION OF THE INVENTION

The extended release tablet dosage form of the present invention is capable of sustaining therapeutic effective for at least twelve hours and also has a Cmax equivalent to that of an immediate release formulation, appears in the blood stream as quickly as an immediate release formulation, yet sustains therapeutic effect for at least twelve hours.

Suitable hydrophilic polymers according to the present invention include polyvinyl pyrrolidone (commercially available as Kollidon®, Plasdone®), copovidone, hydroxymethylcellulose, hydroxyethyl cellulose (Natrosol®), hydroxypropylcellulose (Klucel®), hydroxypropylmethylcellulose (Methocel®), methyl cellulose, polyethylene oxide (Polyox®), polysaccharides such as alginic acid and its salt, xanthan gum and the like or mixtures thereof. The amount of hydrophilic polymer used may be in the range of about 2 to 20% by weight of the tablet.

Suitable water insoluble polymers of the present invention include ethylcellulose (Surelease®), polyacrylic acids, acrylic resins such as carbopol, acrylic latex dispersions, cellulose acetate phthalate (Aquacoat®), polyvinyl acetate phthalate (Phthalavin®, Opaseal®, Sureteric®), hydroxypropyl methylcellulose phthalate (HPMCP®) or mixtures thereof. The amount of water insoluble polymer used may be in the range of about 0.1 to 5% by weight of the tablet.

In yet another preferred embodiment, the extended release formulations further comprise one or more pharmaceutically active compounds selected from antihistamines such as chlorpheniramine, brompheniramine, triprolidine and the like; decongestants such as pseudoephedrine, phenylpropanolamine and the like; antitussives such as caramiphen, dextromethorphan, codeine and the like; expectorants such as terpin hydrate and potassium guaicolsulfonate and the like; non-steroidal anti-inflammatory drugs (NSAIDs) such as ibuprofen, naproxen, piroxicam, indomethacin and the like and analgesics such as phenacetin and acetaminophen.

In another embodiment, the one or more other active compounds may be present either in intragranular portion or extra granular portion or in both portions.

In yet another embodiment, the intragranular portion of the present invention further comprises one or more diluents selected from calcium phosphate-dibasic, calcium silicate, microcrystalline cellulose, lactose, sucrose, pregelatinised starch, polyols such as mannitol, sorbitol, xylitol, maltitol and the like or mixtures thereof.

In yet another embodiment, the extragranular portion of the present invention further comprises one or more pharmaceutically acceptable excipients such as diluents, water insoluble polymers, glidants, lubricants and the like.

Suitable glidants of the present invention include calcium silicate, magnesium carbonate, magnesium oxide, magnesium silicate, colloidal silicon dioxide and the like.

Suitable lubricants of the present invention are selected from sodium stearyl fumarate, magnesium stearate, hydrogenated vegetable oil, stearic acid, calcium stearate, glyceryl behenate, sodium lauryl sulfate, talc and the like or mixtures thereof.

The amount of guaifenesin used according to the present invention is in the range of about 60% to about 90% by weight of the tablet.

In yet another embodiment, the amount of another pharmaceutically active compound, preferably pseudoephedrine or dextromethorphan used may be in the range of 1 to 20% by weight of the total tablet.

In another embodiment, the ratio of hydrophilic polymer to water insoluble polymer is about 1:0.1 to about 10:0.8.

In yet another embodiment, the intragranular portion comprises more than 60% of the total weight of the tablet.

In another embodiment, the present invention also provides a single layer extended release matrix tablet formulation comprising

i) intragranular portion comprising about 60% w/w of guaifenesin, at least one hydrophilic polymer and
ii) extragranular portion comprising hydrophilic polymer and water insoluble polymer,
wherein the ratio of hydrophilic polymer to water insoluble polymer is about 1:0.1 to about 10:0.8.

In a preferred embodiment, there is provide a single layer extended release matrix tablet formulation which comprises

i) intragranular portion comprising

a) about 60% w/w of guaifenesin,

b) at least one hydrophilic polymer selected from the group consisting of polyvinyl pyrrolidone, hydroxyethylcellulose, hydroxypropylcellulose, hydroxypropylmethylcellulose, copovidone,

c) optionally water insoluble polymer and

ii) extragranular portion comprising

a) at least one hydrophilic polymer selected from the group consisting of hydroxyethylcellulose, hydroxypropylcellulose, hydroxypropylmethyl cellulose,

b) optionally guaifenesin, wherein the ratio of hydrophilic polymer in the intra and extra granular portion is about 1:1 to about 1:10.

In another preferred embodiment, there is provide a single layer extended release matrix tablet formulation which comprises

i) intragranular portion comprising

a) about 60% w/w of guaifenesin,

b) at least one hydrophilic polymer selected from the group consisting of polyvinyl pyrrolidone, hydroxyethylcellulose, copovidone, hydroxypropylcellulose, hydroxypropylmethyl cellulose,

c) optionally water insoluble polymer and

ii) extragranular portion comprising

a) guaifenesin and one or more active agents selected from pseudoephedrine, dextromethorphan and phenylpropanolamine,

b) at least one hydrophilic polymer selected from the group consisting of hydroxyethylcellulose, hydroxypropylcellulose, hydroxypropylmethyl cellulose, wherein the ratio of hydrophilic polymer in the intra and extra granular portion is about 1:1 to about 1:10.

In a preferred embodiment, there is provided a single layer extended release matrix tablet formulation, which comprises

i) intragranular portion comprising

a) about 60% w/w of guaifenesin and one or more active agents selected from pseudoephedrine, dextromethorphan and phenylpropanolamine,

b) at least one hydrophilic polymer selected from the group consisting of polyvinyl pyrrolidone, hydroxypropylcellulose, hydroxyethylcellulose, copovidone, hydroxypropylmethyl cellulose,

c) optionally water insoluble polymer and

ii) extragranular portion comprising

a) guaifenesin and one or more active agents selected from pseudoephedrine, dextromethorphan and phenylpropanolamine,

b) at least one hydrophilic polymer selected from the group consisting of hydroxyethylcellulose, hydroxypropylcellulose, hydroxypropylmethyl cellulose, wherein the ratio of hydrophilic polymer in the intra and extra granular portion is about 1:1 to about 1:10.

In another embodiment, there is provided a process for the preparation of single layer extended release matrix tablet of the present invention by dry granulation or wet granulation.

The process of dry granulation comprises the steps of,

i). blending guaifenesin, hydrophilic polymer and optionally water insoluble polymer and other intragranular excipients,
ii). compacting or slugging the blend obtained in step (i),
iii). milling the compacts or slugs obtained in step (ii) and blending with extragranular hydrophilic polymer and optionally guaifenesin and one or more other active agents,
iv). lubricating the blend of step (iii) and
v). compressing the final blend to obtain single layered matrix tablets.

Similarly, the process of wet granulation comprises the steps of,

i). blending guaifenesin, hydrophilic polymer and optionally water insoluble polymer and other intragranular excipients,
ii). granulating the blend of step (i) with suitable solvent followed by drying,
iii). blending the dried granules of step (ii) with extragranular hydrophilic polymer and optionally guaifenesin and one or more other active agents,
iv). lubricating the blend of step (iii) and
v). finally compressing the lubricated blend into single layered tablets.

In an embodiment of the present invention, the solvents used for granulation may be selected from water or organic solvents such as acetone, alcohol, isopropyl alcohol and the mixture thereof.

The following examples further exemplify the invention and are not intended to limit the scope of the invention. It is obvious to those skilled in the art to find out the composition for other dosage forms and substitute the equivalent excipients as described in this specification or with the one known to the industry.

### Example 1

| S. No. | Ingredients | Quantity (mg/tablet) |
|---|---|---|
| | Intragranular | |
| 1. | Guaifenesin | 1203.73 |
| 2. | Pseudoephedrine hydrochloride | 120.63 |
| 3. | Hydroxypropyl methyl cellulose | 20.00 |
| 4. | Magnesium stearate | 6.00 |
| | Extragranular | |
| 5. | Hydroxypropyl methyl cellulose | 50.00 |
| 6. | Microcrystalline cellulose | 193.64 |
| 7. | Magnesium stearate | 6.00 |

The processing steps involved in manufacturing extended release tablets of guaifenesin and pseudoephedrine disclosed in example-1 are given below:

i). guaifenesin, pseudoephedrine and hydroxypropyl methylcellulose were blended,
ii). lubricated the blend of step (i) with magnesium stearate,
iii). prepared the compact of lubricated mass obtained in step (ii),
iv). milled the compacts obtained in step (iii) and blended with extra granular hydroxy propyl methyl cellulose and microcrystalline cellulose,
v). lubricated the blend with magnesium stearate and
vi). compressed the final blend to obtain single layered matrix tablets.

The extended release dosage forms disclosed in examples 2 to 4 were prepared by the similar procedure as described in example 1.

### Example 2

| S. No. | Ingredients | Quantity (mg/tablet) |
|---|---|---|
| | Intragranular | |
| 1. | Guaifenesin | 1143.54 |
| 2. | Pseudoephedrine hydrochloride | 114.6 |
| 3. | Hydroxypropyl methyl cellulose | 20.00 |
| | Extragranular | |
| 4. | Guaifenesin | 60.19 |
| 5. | Pseudoephedrine | 6.03 |
| 6. | Hydroxypropyl methyl cellulose | 60.00 |
| 7. | Carbopol | 6.40 |
| 8. | Microcrystalline cellulose | 177.23 |
| 9. | Magnesium stearate | 12.00 |

### Example 3

| S. No. | Ingredients | Quantity (mg/tablet) |
|---|---|---|
| | Intragranular | |
| 1. | Guaifenesin | 1203.73 |
| 2. | Pseudoephedrine hydrochloride | 120.63 |
| 3. | Hydroxypropyl methyl cellulose | 20.00 |
| 4. | Carbopol | 6.40 |
| | Extragranular | |
| 5. | Hydroxypropyl methyl cellulose | 60.00 |
| 6. | Microcrystalline cellulose | 177.24 |
| 7. | Magnesium stearate | 12.00 |

### Example 4

| S. No. | Ingredients | Quantity (mg/tablet) |
|---|---|---|
| | Intragranular | |
| 1. | Guaifenesin | 1143.54 |
| 2. | Pseudoephedrine hydrochloride | 114.6 |
| 3. | Hydroxypropyl methyl cellulose | 20.00 |
| | Extragranular | |
| 4. | Guaifenesin | 60.19 |
| 5. | Pseudoephedrine | 6.03 |
| 6. | Hydroxypropyl methyl cellulose | 50.00 |
| 7. | Microcrystalline cellulose | 193.64 |
| 8. | Magnesium stearate | 12.00 |

### Example 5

| S. No. | Ingredients | Quantity (mg/tablet) |
|---|---|---|
| | Intragranular | |
| 1. | Guaifenesin | 1143.54 |
| 2. | Pseudoephedrine hydrochloride | 120.6 |
| 3. | Hydroxypropyl methyl cellulose | 20.00 |
| 4. | Carbopol | 6.4 |
| 5. | Water | qs |
| | Extragranular | |
| 6. | Guaifenesin | 60.19 |
| 7. | Hydroxypropyl methyl cellulose | 60.00 |
| 8. | Microcrystalline cellulose | 177.23 |
| 9. | Magnesium stearate | 12.00 |

The processing steps involved in manufacturing extended release tablets of guaifenesin and pseudoephedrine disclosed in example-5 are given below:

i). guaifenesin, pseudoephedrine hydrochloride, hydroxypropyl methylcellulose and carbopol were blended,
ii). granulated the blend of step (i) with water and dried,
iii). dried granules of step (ii) were blended with extragranular guaifenesin to get the homogeneous mixture,
iv). blended the granules of step (iii) with hydroxypropyl methyl cellulose and microcrystalline cellulose,
v). lubricated the blend of step (iv) with magnesium stearate and
vi). compressed the blend of step (v) into single layered tablets.

### Example 6

| S. No. | Ingredients | Quantity (mg/tablet) |
|---|---|---|
| | Intragranular | |
| 1. | Guaifenesin | 1080.00 |
| 2. | Dextromethorphan hydrobromide | 54.00 |
| 3. | Hydroxypropyl methyl cellulose | 20.00 |
| 4. | Water | q.s |
| | Extragranular | |
| 5. | Guaifenesin | 120.00 |
| 6. | Dextromethorphan hydrobromide | 6.00 |
| 7. | Hydroxypropyl methyl cellulose | 60.00 |
| 8. | Carbopol | 6.4 |
| 9. | Microcrystalline cellulose | 113.6 |
| 10. | Magnesium stearate | 15.00 |

The processing steps involved in manufacturing extended release tablets of guaifenesin and dextromethorphan hydrobromide disclosed in example-6 are given below:

i). guaifenesin, dextromethorphan hydrobromide and hydroxypropyl methylcellulose were blended,
ii). granulated the blend of step (i) with water and dried,
iii). dried granules of step (ii) were blended with extragranular guaifenesin and dextromethorphan hydrobromide to get the homogeneous mixture,
iv). blended the granules of step (iii) with hydroxypropyl methyl cellulose, carbopol and microcrystalline cellulose,
v). lubricated the blend of step (iv) with magnesium stearate and
vi). compressed the blend of step (v) into single layered tablets.

### Example 7

| S. No. | Ingredients | Quantity (mg/tablet) |
|---|---|---|
| | Intragranular | |
| 1. | Guaifenesin | 1200.00 |
| 2. | Pregelatinised starch | 160.00 |
| 3. | Povidone | 20.00 |
| 4. | Water | q.s |
| | Extragranular | |
| 5. | Dextromethorphan hydrobromide | 60.00 |
| 6. | Hydroxypropyl methyl cellulose | 78.00 |
| 7. | Carbopol | 6.00 |
| 8. | Microcrystalline cellulose | 59.50 |
| 9. | Colloidal silicon dioxide | 8.25 |
| 10. | Stearic acid | 8.25 |

The processing steps involved in manufacturing extended release tablets of guaifenesin and dextromethorphan hydrobromide disclosed in example-7 are given below:

i). guaifenesin, pregelatinised starch were blended,
ii). granulated the blend of step (i) with aqueous solution of povidone and dried,
iii). dried granules of step (ii) were blended with extragranular dextromethorphan hydrobromide, hydroxypropylmethylcellulose, carbopol, microcrystalline cellulose and colloidal silicon dioxide,
iv). lubricated the blend of step (iii) with stearic acid and
v). compressed the blend of step (iv) into single layered tablets.

The extended release dosage forms disclosed in examples 8 to 10 were prepared by the similar procedure as described in example 7.

### Example 8

| S. No. | Ingredients | Quantity (mg/tablet) |
|---|---|---|
| | Intragranular | |
| 1. | Guaifenesin | 1200.00 |
| 2. | Pregelatinised starch | 160.00 |
| 3. | Copovidone | 20.00 |
| 4. | Water | q.s |
| | Extragranular | |
| 5. | Dextromethorphan hydrobromide | 60.00 |
| 6. | Hydroxypropyl methyl cellulose | 65.00 |
| 7. | Carbopol | 5.00 |
| 8. | Microcrystalline cellulose | 60.00 |
| 9. | Colloidal silicon dioxide | 8.00 |
| 10. | Stearic acid | 12.00 |

### Example 9

| S. No. | Ingredients | Quantity (mg/tablet) |
|---|---|---|
| | Intragranular | |
| 1. | Guaifenesin | 1200.00 |
| 2. | Pregelatinised starch | 160.00 |
| 3. | Hydroxypropylcellulose | 20.00 |
| 4. | Water | q.s |
| | Extragranular | |
| 5. | Pseudoephedrine hydrochloride | 120.00 |
| 6. | Hydroxypropyl methyl cellulose | 65.00 |
| 7. | Carbopol | 5.00 |
| 8. | Microcrystalline cellulose | 65.00 |
| 9. | Colloidal silicon dioxide | 8.00 |
| 10. | Stearic acid | 12.00 |

### Example 10

| S. No. | Ingredients | Quantity (mg/tablet) |
|---|---|---|
| | Intragranular | |
| 1. | Guaifenesin | 1200.00 |
| 2. | Pregelatinised starch | 160.00 |
| 3. | Hydroxypropylmethylcellulose | 20.00 |
| 4. | Water | q.s |
| | Extragranular | |
| 5. | Pseudoephedrine hydrochloride | 120.00 |
| 6. | Hydroxypropyl methyl cellulose | 65.00 |
| 7. | Carbopol | 5.00 |
| 8. | Microcrystalline cellulose | 65.00 |
| 9. | Colloidal silicon dioxide | 8.00 |
| 10. | Stearic acid | 12.00 |

### Example 11

| S. No. | Ingredients | Quantity (mg/tablet) |
|---|---|---|
| | Intragranular | |
| 1. | Guaifenesin | 1200.00 |
| 2. | Pseudoephedrine hydrochloride | 120 |
| 3. | Pregelatinised starch | 160.00 |
| 4. | Copovidone | 20.00 |
| 5. | Water | q.s |
| | Extragranular | |
| 6. | Hydroxypropyl methyl cellulose | 65 |
| 7. | Carbopol | 5.00 |
| 8. | Microcrystalline cellulose | 65 |
| 9. | Colloidal silicon dioxide | 8.00 |
| 10. | Stearic acid | 12 |

The processing steps involved in manufacturing extended release tablets of guaifenesin and pseudoephedrine hydrochloride disclosed in example-11 are given below:

i). guaifenesin, pseudoephedrine hydrochloride, pregelatinised starch, copovidone were blended,
ii). granulated the blend of step (i) with water and dried,
iii). dried granules of step (ii) were blended with extragranular hydroxypropylmethylcellulose, carbopol, microcrystalline cellulose and colloidal silicon dioxide,
iv). lubricated the blend of step (iii) with stearic acid and
v). compressed the blend of step (iv) into single layered tablets.

The extended release dosage forms disclosed in examples 12 to 16 were prepared by the similar procedure as described in example 11.

### Example 12

| S. No. | Ingredients | Quantity (mg/tablet) |
|---|---|---|
| | Intragranular | |
| 1. | Guaifenesin | 1200.00 |
| 2. | Pseudoephedrine hydrochloride | 120 |
| 3. | Pregelatinised starch | 160.00 |
| 4. | Hydroxypropylmethylcellulose | 20.00 |
| 5. | Water | q.s |
| | Extragranular | |
| 6. | Hydroxypropyl methyl cellulose | 65 |
| 7. | Carbopol | 5.00 |
| 8. | Microcrystalline cellulose | 65 |
| 9. | Colloidal silicon dioxide | 8.00 |
| 10. | Stearic acid | 12 |

### Example 13

| S. No. | Ingredients | Quantity (mg/tablet) |
|---|---|---|
| | Intragranular | |
| 1. | Guaifenesin | 1200.00 |
| 2. | Dextromethorphan hydrobromide | 60 |
| 3. | Pregelatinised starch | 160.00 |
| 4. | Hydroxypropylcellulose | 20.00 |
| 5. | Water | q.s |
| | Extragranular | |
| 6. | Hydroxypropyl methyl cellulose | 65 |
| 7. | Carbopol | 5.00 |
| 8. | Microcrystalline cellulose | 60 |
| 9. | Colloidal silicon dioxide | 8.00 |
| 10. | Stearic acid | 12 |

### Example 14

| S. No. | Ingredients | Quantity (mg/tablet) |
|---|---|---|
| | Intragranular | |
| 1. | Guaifenesin | 1200.00 |
| 2. | Pregelatinised starch | 240.50 |
| 3. | Hydroxyethylcellulose | 17.50 |
| 4. | Water | q.s |
| | Extragranular | |
| 5. | Dextromethorphan hydrobromide | 60.00 |
| 6. | Hydroxypropyl methyl cellulose | 65.00 |
| 7. | Carbopol | 5.00 |
| 8. | Colloidal silicon dioxide | 4.00 |
| 9. | Stearic acid | 8.00 |

### Example 15

| S. No. | Ingredients | Quantity (mg/tablet) |
|---|---|---|
| | Intragranular | |
| 1. | Guaifenesin | 1200.00 |
| 2. | Pregelatinised starch | 160.00 |
| 3. | Povidone | 17.50 |
| 4. | Water | q.s |
| | Extragranular | |
| 5. | Dextromethorphan hydrobromide | 60.00 |
| 6. | Hydroxypropyl methyl cellulose | 65.00 |
| 7. | Carbopol | 5.00 |
| 8. | Microcrystalline cellulose | 76.50 |
| 9. | Colloidal silicon dioxide | 8.00 |
| 10. | Stearic acid | 8.00 |

### Example 16

| S. No. | Ingredients | Quantity (mg/tablet) |
|---|---|---|
| | Intragranular | |
| 1. | Guaifenesin | 1200.00 |
| 2. | Pseudoephedrine hydrochloride | 120.00 |
| 3. | Pregelatinised starch | 160.00 |
| 4. | Povidone | 17.50 |
| 5. | Water | q.s |
| | Extragranular | |
| 6. | Hydroxypropyl methyl cellulose | 65.00 |
| 7. | Carbopol | 5.00 |
| 8. | Microcrystalline cellulose | 66.50 |
| 9. | Colloidal silicon dioxide | 8.00 |
| 10. | Stearic acid | 8.00 |

The dissolution profile of the extended release tablets of Guaifenesin prepared acording to the present inveniton were carried out in 900 ml of 0.1N hydrochloric acid as medium according to the procedure described in the USP, Apparatus USP I/900 ml, Basket, @ 75 rpm speed. The release profile (% of drug released in minutes) is given in tables 1-2.

TABLE 1

| | |
|---|---|
| Time | % drug dissolved (Example 15) |

| (hours) | Guaifenesin | Dextromethorphan |
|---|---|---|
| 1 | 42 | 36 |
| 2 | 56 | 53 |
| 4 | 73 | 72 |
| 6 | 85 | 85 |
| 8 | 95 | 94 |

TABLE 2

| | |
|---|---|
| Time | % drug dissolved (Example 16) |

| (hours) | Guaifenesin | Pseudoephedrine |
|---|---|---|
| 1 | 42 | 52 |
| 2 | 56 | 67 |
| 4 | 75 | 85 |
| 6 | 88 | 96 |
| 8 | 99 | 104 |

We claim:

1. A single layer extended release matrix tablet formulation comprising
i) intragranular portion comprising about 60% w/w of guaifenesin, at least one hydrophilic polymer and optionally a water insoluble polymer and
ii) extragranular portion comprising hydrophilic polymer and optionally guaifenesin,
wherein the ratio of hydrophilic polymer in the intra and extra granular portion is about 1:1 to about 1:10.

2. The extended release tablet of claim 1, wherein the hydrophilic polymer is selected from the group consisting of polyvinyl pyrrolidone, hydroxymethyl cellulose, copovidone, hydroxyethylcellulose, hydroxypropylcellulose, hydroxypropylmethylcellulose, methyl cellulose, polyethylene oxide, alginic acid and its salt, xanthan gum or mixture thereof.

3. The extended release tablet of claim 1, wherein the water insoluble polymer is selected from the group consisting of ethylcellulose, carbopol, cellulose acetate phthalate, polyvinyl acetate phthalate, hydroxypropylmethylcellulose phthalate or mixture thereof.

4. The extended release tablet of claim 1, further comprise one or more pharmaceutically active compounds selected from chlorpheniramine, brompheniramine, triprolidine, pseudoephedrine, phenylpropanolamine, caramiphen, dextromethorphan and codeine.

5. The extended release tablet of claim 1, wherein the extragranular portion further comprises one or more excipients selected from diluent, water insoluble polymer, glidant and lubricant.

6. The extended release tablet of claim 5, wherein the diluent is selected from calcium phosphate-dibasic, calcium silicate, microcrystalline cellulose, lactose, sucrose, pregelatinised starch, mannitol, sorbitol, xylitol, maltitol or mixture thereof.

7. The extended release tablet of claim 5, wherein the glidant is selected from calcium silicate, magnesium carbonate, magnesium oxide, magnesium silicate and colloidal silicon dioxide.

8. The extended release tablet of claim 5, wherein the lubricant is selected from sodium stearyl fumarate, magnesium stearate, hydrogenated vegetable oil, stearic acid, calcium stearate, glyceryl behenate, sodium lauryl sulfate and talc.

9. The extended release tablet of claim 1, wherein the ratio of hydrophilic polymer to water insoluble polymer is about 1:0.1 to about 10:0.8.

10. The extended release tablet of claim 1, wherein the amount of hydrophilic polymer is in the range of about 2 to 20% by weight of the tablet.

11. The extended release tablet of claim 1, wherein the amount of water insoluble polymer is in the range of about 0.1 to 5% by weight of the tablet.

12. A single layer extended release matrix tablet formulation comprising
i) intragranular portion comprising
a) about 60% w/w of guaifenesin,
b) at least one hydrophilic polymer
c) optionally water insoluble polymer and
ii) extragranular portion comprising
a) guaifenesin and one or more active agents selected from pseudoephedrine, dextromethorphan and phenylpropanolamine,
b) at least one hydrophilic polymer,
wherein the ratio of hydrophilic polymer in the intra and extra granular portion is about 1:1 to about 1:10.

13. The extended release tablet of claim 12, wherein the hydrophilic polymer is selected from the group consisting of polyvinyl pyrrolidone, hydroxymethyl cellulose, copovidone, hydroxyethylcellulose, hydroxypropylcellulose, hydroxypropylmethylcellulose, methyl cellulose, polyethylene oxide, alginic acid and its salt, xanthan gum or mixture thereof.

14. The extended release tablet of claim 12, wherein the water-insoluble polymer is selected from the group consisting of ethylcellulose, carbopol, cellulose acetate phthalate, polyvinyl acetate phthalate, hydroxypropylmethylcellulose phthalate or mixture thereof.

15. The extended release tablet of claim 12, wherein the extragranular portion further comprise one or more excipients selected from diluent, water insoluble polymer, glidant and lubricant.

16. The extended release tablet of claim 15, wherein the diluent is selected from calcium phosphate-dibasic, calcium silicate, microcrystalline cellulose, lactose, sucrose, pregelatinised starch, mannitol, sorbitol, xylitol, maltitol or mixture thereof.

17. A single layer extended release matrix tablet formulation comprising
i) intragranular portion comprising
a) about 60% w/w of guaifenesin and one or more active agents selected from pseudoephedrine, dextromethorphan and phenylpropanolamine,
b) at least one hydrophilic polymer,
c) optionally water insoluble polymer and
ii) extragranular portion comprising
a) guaifenesin and one or more active agents selected from pseudoephedrine, dextromethorphan and phenylpropanolamine,
b) at least one hydrophilic polymer,
wherein the ratio of hydrophilic polymer in the intra and extra granular portion is about 1:1 to about 1:10.

18. The extended release tablet of claim 17, wherein the hydrophilic polymer is selected from the group consisting of polyvinyl pyrrolidone, hydroxymethyl cellulose, copovidone, hydroxyethylcellulose, hydroxypropylcellulose, hydroxypropylmethylcellulose, methyl cellulose, polyethylene oxide, alginic acid and its salt, xanthan gum or mixture thereof.

19. The extended release tablet of claim 17, wherein the water-insoluble polymer is selected from the group consisting of ethylcellulose, carbopol, cellulose acetate phthalate, polyvinyl acetate phthalate, hydroxypropylmethylcellulose phthalate or mixture thereof.

20. The extended release tablet of claim 17, wherein the extragranular portion further comprises one or more excipients selected from diluent, water insoluble polymer, glidant and lubricant.
